(19) United States
(12) Patent Application Publication
(10) Pub. No.: US 2023/0338004 A1
(43) Pub. Date: Oct. 26, 2023

(54) ULTRASOUND TISSUE DIFFERENTIATION SYSTEM

(71) Applicant: NINA MEDICAL LTD., Nazareth (IL)

(72) Inventors: Shmuel BEN EZRA, Pardes-Hanna (IL); Yoni HERTZBERG, Shoham (IL); Doron KOPELMAN, Caesarea (IL); Alexander VOLOVICK, Rishon LeZion (IL)

(21) Appl. No.: 18/026,691

(22) PCT Filed: Sep. 14, 2021
(86) PCT No.: PCT/IL2021/051120

§ 371 Date: Mar. 16, 2023

Related U.S. Application Data
(60) Provisional application No. 63/079,485, filed on Sep. 17, 2020.

(51) Int. Cl. A61N 7/02; A61B 8/08; A61B 8/00; G01S 15/89

(52) U.S. Cl. CPC A61B 8/085; A61B 8/4477; A61B 8/463; A61B 8/488; A61N 7/02; G01S 15/8952; A61N 2007/0073

(57) ABSTRACT

Apparatus (18) assesses a characteristic of a tissue (46). A set (80) of one or more acoustic transducers (50, 54) transmits a first acoustic field (48) at a first frequency into the tissue, generating oscillatory motion at the first frequency of scatterers disposed in the tissue. A second acoustic field (56) at a second frequency higher than the first frequency is transmitted into the tissue. Echo data is received due to the second acoustic field scattering off an oscillating scatterer that is oscillating at the first frequency. A computer processor (29) derives an indication of acoustic impedance of the tissue based on the echo data, and drive an output device (40) to output an indication of whether the tissue is or may be a tumor, based on the indication of the acoustic impedance. Other embodiments are also described.

### CROSS REFERENCE TO RELATED APPLICATIONS

The present application claims the priority of US 63/079,485 to Ben Ezra et al., filed Sep. 17, 2020, entitled, “Ultrasound cancer detection system,” which is incorporated herein by reference.

### FIELD OF THE INVENTION

The present invention relates to the medical field, in particular to the field of medical imaging as it pertains to the differentiation of tissue structures, for example, for cancer detection.

### BACKGROUND OF THE INVENTION

Ultrasound energy is often used for imaging of internal organs and tissue. High intensity focused ultrasound (HIFU), also known as high intensity therapeutic ultrasound (HITU), is a method for non-invasive treatment of internal organs and tissue, e.g., tumors. Ultrasound has been used for tissue differentiation, e.g., by measuring echogenicity (brightness of tissue in a standard ultrasound image), and morphology of tissue (e.g., a tumor), for example by detecting contours (edges), surface texture, blood vessels, and other characteristics.

Acoustic radiation force imaging (ARFI) has been described for use to find the elasticity of tissue at low frequencies, based on an assumption that this elasticity is different in cancerous vs. normal tissue, for certain types of cancers. An NIH Public Access review article dated Nov. 1, 2011, entitled “Acoustic radiation force impulse (ARFI) imaging: A review,” by Kathy Nightingale, describes acoustic radiation force-based elasticity imaging methods that are under investigation by many groups. Methods have been developed that utilize impulsive (i.e., < 1 ms), harmonic (pulsed), and steady state radiation force excitations. The work discussed in the review article utilizes impulsive methods, for which two imaging approaches have been pursued: 1) monitoring the tissue response within the radiation force region of excitation (ROE) and generating images of relative differences in tissue stiffness (Acoustic Radiation Force Impulse (ARFI) imaging); and 2) monitoring the speed of shear wave propagation away from the ROE to quantify tissue stiffness (Shear Wave Elasticity Imaging (SWEI)). For these methods, a single ultrasound transducer on a commercial ultrasound system is described as being used to both generate acoustic radiation force in tissue, and to monitor the tissue displacement response. The response of tissue to this transient excitation is described as being complicated and depending upon tissue geometry, radiation force field geometry, and tissue mechanical and acoustic properties. Higher shear wave speeds and smaller displacements are associated with stiffer tissues, and slower shear wave speeds and larger displacements occur with more compliant tissues. ARFI imaging is described in the article as having spatial resolution comparable to that of B-mode, often with greater contrast, providing matched, adjunctive information. SWEI images are described as having quantitative information about the tissue stiffness, typically with lower spatial resolution.

In an article by Duzgun et al., entitled “Is computed tomography perfusion a useful method for distinguishing between benign and malignant neck masses?” (ENT Journal, Volume 96(6)), the authors state that evaluation of neck masses is frequent in ear, nose, and throat clinics. Successful outcomes associated with neck masses are described as being directly related to rapid diagnosis and accurate treatment for each patient. Late diagnosis of a malignant mass is described as increasing the magnitude of morbidity and the rate of mortality of the disease. Although magnetic resonance imaging and computed tomography (CT) examinations are described as important tools for evaluating head and neck pathologies, they are described as not allowing functional evaluation. For this reason, the authors describe CT perfusion (CTP) as a method that is gaining attention for functional evaluation for distinguishing benign from malignant masses. The utility of CTP for distinguishing between benign and malignant mass lesions was investigated in 35 patients with masses in the neck (11 benign, 24 malignant). CTP is described by the authors as having been shown to be a useful method for identifying head and neck tumors, and blood volume values are described by the authors as having been shown to enable the differential diagnosis of benign and malignant head and neck tumors.

US 2019/0232090 to Ben-Ezra, which is incorporated herein by reference, describes apparatus for assessing a characteristic of a first acoustic field at a first frequency in a region of a medium, the first acoustic field generating oscillatory motion at the first frequency of scatterers disposed within the medium. An acoustic transducer (a) generates a second acoustic field at a second frequency in the region, the second frequency being higher than the first frequency, and (b) receives echo data of the second acoustic field scattering off the oscillating scatterers in the medium. The echo data contains Doppler-shifted frequencies related to the oscillations of the scatterers, resulting in a time-dependent Doppler shift that oscillates at a frequency that is related to the first frequency. Control circuitry (a) extracts the oscillating time-dependent Doppler shift from the received echo data, and (b) converts the extracted Doppler shift into particle-velocity of the first acoustic field.

US 2021/0045714 to Ben-Ezra, which is incorporated herein by reference, describes a first transducer that transmits a first acoustic field at a first frequency into a region of a medium, generating oscillatory motion of scatterers disposed in the region. A second transducer transmits acoustic pulses into the region, and receives respective echoes of each pulse scattering off an oscillating scatterer in the region. The pulses are synchronized with the first acoustic field such that a first pulse scatters off the oscillating scatterer when the scatterer is at a first displacement extremum, and a second pulse scatters off the oscillating scatterer when the scatterer is at a second displacement extremum that is opposite the first displacement extremum. A computer processor extracts a time shift between the received echoes, calculates a displacement amplitude of the scatterer, and outputs an indication of the displacement amplitude of the scatterer.

### SUMMARY OF THE INVENTION

In accordance with some applications of the present invention, apparatus and methods are provided for detecting an indication of whether a tissue is or may be a tumor, based on an indication of the acoustic impedance of the tissue. The acoustic impedance is determined by transmitting two acoustic fields into the tissue. The first acoustic field is transmitted at a first frequency, and generates oscillatory motion at the first frequency of scatterers disposed in the tissue. A second acoustic field is transmitted at a second frequency into the tissue, the second frequency being higher than the first frequency. Typically, neither field causes any therapeutic heating of the tissue -- indeed, the fields typically (but not necessarily) cause little or no tissue heating whatsoever. Echo data is received by a transducer due to the second acoustic field scattering off an oscillating scatterer in the tissue that is oscillating at the first frequency, and a computer processor derives an indication of the acoustic impedance of the tissue based on the echo data.

The acoustic impedance of the tissue may be output numerically or using various image display techniques that are known in the art of ultrasound. For some applications, an image of the acoustic impedance of the tissue in a plane of the tissue is displayed, optionally fused with or side-by-side with an image of the anatomy of the tissue (e.g., in the same plane) to facilitate an assessment of the tissue. For example, the assessment may be performed as part of a screening procedure, and in some cases, may indicate that the tissue should be considered in a follow up procedure (e.g., a biopsy or another imaging procedure, such as a CT scan or an MRI) based on the acoustic impedance. Alternatively, for some applications, a therapy (e.g., high-intensity focused ultrasound (HIFU)) may be initiated based on the determination described above.

For some applications, the applications of the present invention described herein provide a new map, which, while useful for distinguishing cancerous tissue from non-cancerous tissue, may also be used to identify other differences between tissues in a patient based on the characteristic of acoustic impedance.

Data collection (e.g., imaging, e.g., HIFU imaging) using the two acoustic fields described herein may be used to determine acoustic impedance of tissue. For some applications, temporal change and/or spatial variations or gradients of acoustic impedance are tracked or identified using this method, allowing for an acoustic impedance image or other output modality to be formed and for this image or other modality to be tracked over time, e.g., over two minutes during thermal ablation of the tissue, or during sub-ablating heating of tissue (for example, heating of the tissue by less than 5° C.). This tracked acoustic impedance information is useful in distinguishing normal from cancerous tissue.

For some applications, focused ultrasound (e.g., HIFU) is used to heat tissue, and imaging methods (e.g., as described herein using two acoustic fields) are used to measure temperature dependent changes in the acoustic impedance of the tissue, allowing for determination of a thermal response of the tissue. This thermal response is in general different for cancerous tissue as compared to noncancerous tissue. The thermal response of a given mass of tissue depends on several factors including the perfusion of the tissue, i.e., the rate of blood flow through the tissue (high perfusion hinders heating and promotes cooling and vice versa), the heat capacity of the tissue, the heat conductivity of the tissue, and the nature of the surrounding tissue and thermal coupling thereto. For example, a heating modality, e.g., an ultrasound field, e.g., a HIFU field, may be applied to heat the tissue, and a computer processor may track the temporal variation of the impedance as the tissue temperature rises and/or returns toward a baseline value. The perfusion of blood is higher in cancerous tissue than in non-cancerous tissue, and therefore different characteristics of tissue may be identified, in accordance with these applications of the present invention, using techniques for detecting changes in acoustic impedance as described herein.

For some applications, one or more methods are practiced that are used alone or in combination for detection of cancerous masses. For some applications, two or more of the methods described herein are combined after suitable registration to provide a clearer image of cancerous versus noncancerous tissue than is possible with any of them alone. In a particular application, all are executed using the same transducer unit, e.g., a combined HIFU/imaging transducer.

The present invention will be more fully understood from the following detailed description of applications thereof, taken together with the drawings, in which:

### BRIEF DESCRIPTION OF THE DRAWINGS

FIG. 1 is a schematic illustration of apparatus for assessing a characteristic of a medium, such as a tissue of a subject, in accordance with some applications of the present invention;

FIGS. 2A, 2B, and 2C are schematic illustrations showing various configurations of a first acoustic transducer, a second acoustic transducer, and an imaging transducer, in accordance with respective applications of the present invention; and

FIG. 3 is a schematic illustration of an acoustic transducer system, in accordance with some applications of the present invention.

### DETAILED DESCRIPTION OF APPLICATIONS

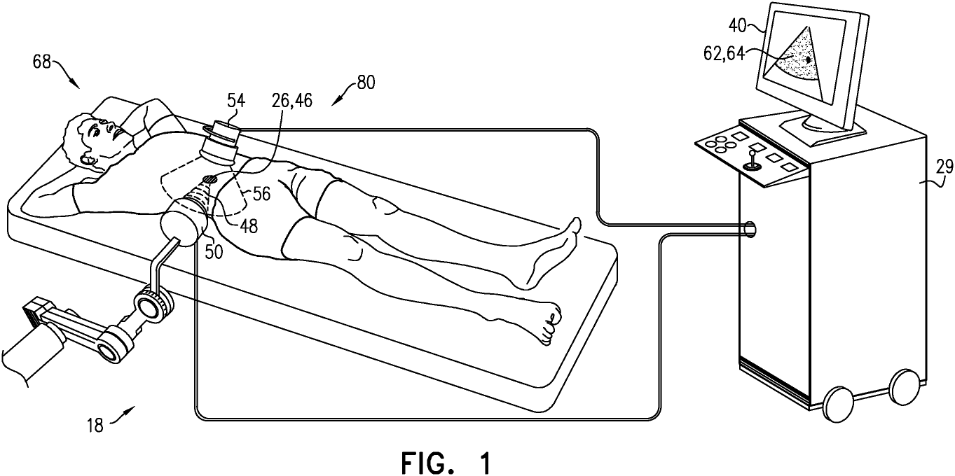

FIG. 1 is a schematic illustration of apparatus 18 for assessing a characteristic of a medium 26, such as a tissue 46 of a subject 68, in accordance with some applications of the present invention. The apparatus comprises a set 80 of one or more acoustic transducers 50, 54 that transmits a first acoustic field 48 at a first frequency into tissue 46. The first acoustic field generates oscillatory motion at the first frequency of scatterers disposed in the tissue, each scatterer oscillating around a respective equilibrium position. In addition, set 80 of acoustic transducers transmits into tissue 46 a second acoustic field 56 at a second frequency, the second frequency higher than the first frequency. Set 80 of acoustic transducers receives echo data due to second acoustic field 56 scattering off an oscillating scatterer in the tissue that is oscillating at the first frequency. Apparatus 18 additionally comprises at least one computer processor 29 that derives an indication of acoustic impedance of tissue 46 based on the echo data, and drives an output device 40 (such as a computer monitor) to output an indication of whether tissue 46 is or may be a tumor, or whether tissue 46 is or may be a malignant tumor versus a benign tumor, based on the indication of the acoustic impedance of the tissue.

For some applications, apparatus 18 is used as a tool in an overall information gathering regime regarding subject 68, rather than in order to provide a diagnosis that is immediately actionable (e.g., such that the next step in treating subject 68 would be to ablate an identified tumor). Thus, for example, set 80 of acoustic transducers may perform the steps of transmitting first acoustic field 48, transmitting second acoustic field 56, and receiving the echo data without therapeutically heating tissue 46 as part of the procedure described above or subsequently thereto. (Indeed, as described hereinbelow, apparatus 18 may not cause significant heating of tissue 46 during the procedure described above, or even any heating at all.) Correspondingly, for some applications, a physician may use information derived using apparatus 18 as a justification for performing a biopsy subsequently to outputting the indication of whether tissue 46 is or may be a tumor. As appropriate, the biopsy may be performed under ultrasound guidance in the same procedure described above in which the first and second acoustic fields are transmitted.

Similarly, computer processor 29 may drive output device 40 to display absolute values related to the acoustic impedance of the tissue (perhaps after normal image processing steps, such as noise reduction), and these absolute values are not displayed relative to standard (e.g., population-derived) values of acoustic impedance. Thus, for example, output device 40 may display the absolute values as an acoustic-impedance image 64, wherein respective pixel values in the acoustic-impedance image are indicative of respective acoustic impedance values at different spatial locations within the tissue. (Alternatively or additionally, in a mode of operation of computer processor 29, output device 40 displays values related to the acoustic impedance of the tissue that are relative to standard values for acoustic impedance.)

Regardless of how acoustic-impedance image 64 is generated (with respect to absolute or relative values), respective pixel values in the image 64 are typically indicative of respective acoustic impedance values at different spatial locations within tissue 46. Typically, set 80 of acoustic transducers additionally transmits an imaging acoustic field into tissue 46 (e.g., at 2-12 MHz), and output device 40 displays an anatomical image 62 (e.g., a B-mode image) of tissue 46 based on echo data from the imaging acoustic field, optionally fusing the acoustic-impedance image with anatomical image 62. It is hypothesized that displaying acoustic-impedance image 64 (typically in addition to and/or fused with anatomical image 62) as provided herein is advantageous. For example, in the liver, B-mode imaging often yields a large, generally grey area on the display due to the generally homogeneous reflectivity of the liver, even if there is a tumor in the liver. Acoustic-impedance image 64 is expected to be less homogeneous, particularly when it includes tumor and non-tumor tissue.

For some applications, the first acoustic field is transmitted at a frequency of at least 1 MHz (e.g., 1-2.5 MHz), for example as high intensity focused ultrasound (HIFU), particularly in cases where tissue heating is desired, as described hereinbelow.

Alternatively (for example when tissue heating is not necessary), set 80 of acoustic transducers sets the first frequency to be less than 1 MHz, e.g., less than 500 kHz. For applications in which the first frequency is set at 100-500 kHz, the subject is typically generally unable to hear or otherwise sense the first acoustic field. For applications in which the first frequency is set at 20-50 kHz or perhaps 50-100 kHz, the procedure described herein can be successfully performed, however the subject may be able to sense the first acoustic field. With these lower frequencies, first acoustic field 48 is typically less focused and less collimated than a high intensity focused ultrasound field.

The second frequency is typically set to be 2-12 MHz, and/or to be 2-50 times higher (e.g., 2-10 or 10-50 times higher) than the first frequency.

Typically, set 80 of acoustic transducers inhibits heating of tissue 46 by controlling a time-averaged intensity (or another measure of intensity) of first acoustic field 48. Correspondingly, computer processor 29 typically derives the indication of the acoustic impedance irrespective of any change in tissue 46 due to any temperature rise of the tissue that might be induced by first acoustic field 48. In order to inhibit heating of tissue 46, the time between the initiation of successive pulses of ultrasound energy in first acoustic field 48 is typically set to be 20-100 times longer or even 100-500 times longer than an average pulse duration of the successive pulses of the ultrasound energy. Alternatively or additionally, a duty cycle of first acoustic field 48 is kept low (e.g., to less than 1%), and the oscillatory motion of the scatters is generated by transmitting only for example 1-15 cycles of the first acoustic field in order to inhibit tissue heating. Further alternatively or additionally, the pulse repetition frequency (PRF) of first acoustic field 48 is kept low, for example, to under 50 Hz (e.g., 5-50 Hz, e.g., 10-25 Hz) in order to inhibit tissue heating. The amplitude of the first acoustic field may be set to be less than 5 MPa (typically higher than 0.1 MPa).

Using one or more (or all) of these parameters, the time-averaged intensity of the first acoustic field may be controlled to be less than approximately 720 mW/cm^2. In this manner, set 80 of acoustic transducers typically prevents any therapeutic heating of tissue 46, and for some applications prevents any noticeable heating of the tissue. (For example, any heating of the tissue may be limited to 2° C., or 1 degree C.)

Alternatively, set 80 configures first acoustic field 48 to heat tissue 46 (e.g., by at least 1 degree C, e.g., by 2-5° C., e.g., by 2-3° C.) from a first temperature, for example by configuring first acoustic field 48 to be a HIFU field, and computer processor 29 derives the indication of the acoustic impedance at a plurality of time points following initiation of the heating of the tissue, while the tissue is at respective temperatures elevated above the first temperature. A temporal separation between at least one of the plurality of time points and another one of the plurality of time points is typically set to be at least 20 and/or less than 500 milliseconds (e.g., 20-500 milliseconds, e.g., 100 milliseconds), and computer processor 29 typically distributes the plurality of time points over at least 5 seconds, e.g., 30-120 seconds.

For some applications, at least one time point of the plurality of time points is set to be following termination of the heating of the tissue, i.e., while tissue 46 is cooling following the heating of the tissue. Alternatively or additionally, at least one time point of the plurality of time points is set to be following initiation of the heating and prior to termination of the heating of the tissue, i.e., while the temperature of tissue 46 is increasing during heating. Thus, using the techniques described herein, the difference in blood perfusion between tumor tissue and non-tumor tissue, and between malignant and non-malignant tumor tissue, is detectable, because the slower heating and slower cooling of tumors (and in particular malignant tumors) yields a slower change in acoustic impedance than in healthy tissue. For some applications, acoustic impedance data derived at some or all of the plurality of time points are compared to baseline acoustic impedance data derived prior to the initiation of heating, using the techniques described herein.

For some applications, a therapeutic acoustic field, e.g., a HIFU field, is transmitted into tissue 46, subsequently to the driving of output device 40 and at least in part in response to the derived indication of the acoustic impedance of tissue 46. The therapeutic acoustic field has an intensity that is higher (e.g., 100 times higher) than an intensity of the first acoustic field and that is higher (e.g., 100 times higher) than an intensity of the second acoustic field. In a particular application of the present invention, a single ultrasound transducer (first acoustic transducer 50) is used for transmitting first acoustic field 48 and the therapeutic acoustic field. Typically in this case, second acoustic field 56 is transmitted using a different ultrasound transducer (namely, second acoustic transducer 54) from that which is used to transmit the first acoustic field and the therapeutic acoustic field.

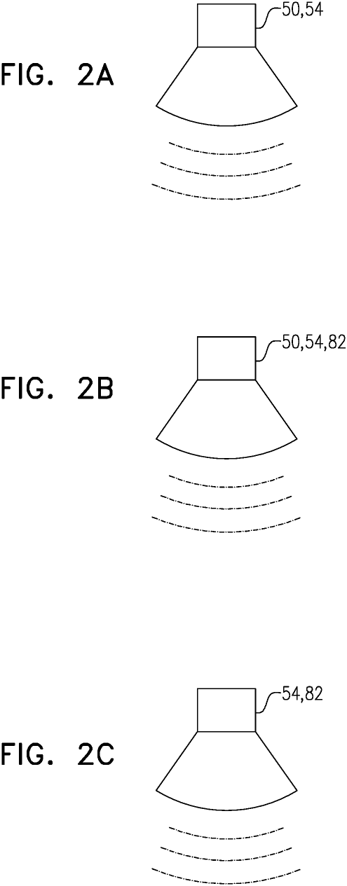

Reference is now made to FIGS. 2A, 2B, and 2C, which show various configurations of first acoustic transducer 50, which transmits first acoustic field 48, second acoustic transducer 54, which transmits second acoustic field 56, and an imaging transducer 82, in accordance with respective applications of the present invention. By contrast to FIG. 1, which shows first and second acoustic transducers 50 and 54 as discrete units, FIG. 2A shows a single acoustic transducer (e.g., a single ultrasound transducer) for transmitting the first and second acoustic fields. FIG. 2B shows a single acoustic transducer (e.g., a single ultrasound transducer) serving as first acoustic transducer 50, second acoustic transducer 54, and imaging transducer 82, and configured to transmit first acoustic field 48, second acoustic field 56, and the imaging acoustic field.

FIG. 2C shows a single acoustic transducer (e.g., a single ultrasound transducer) serving as second acoustic transducer 54 and imaging transducer 82, for transmitting second acoustic field 56 and the imaging acoustic field. In this context, first acoustic transducer 50 (serving as a first-acoustic-field transducer, e.g., configuring the first acoustic field as an ultrasound field) is distinct from the single acoustic transducer (e.g., the single ultrasound transducer) shown in FIG. 2C.

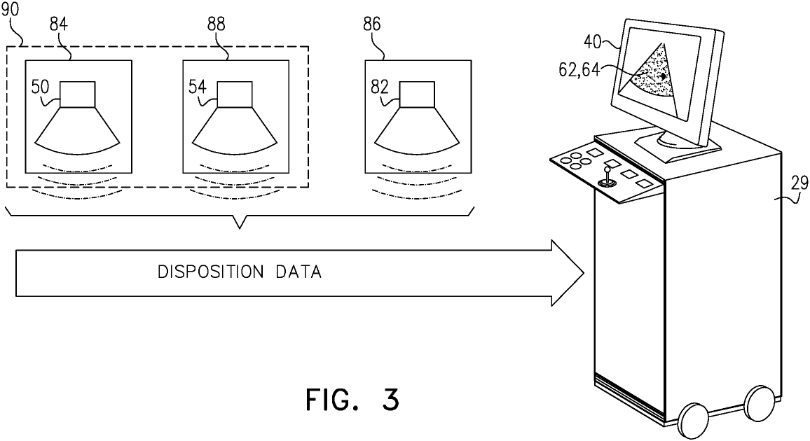

Reference is now made to FIG. 3, which is a schematic illustration of an acoustic transducer system, in accordance with some applications of the present invention. Imaging transducer 82 transmits the imaging acoustic field and second acoustic transducer 54 (serving as a second-acoustic-field transducer) transmits the second acoustic field. Imaging transducer 82 is disposed in an imaging-transducer housing 86, and second acoustic transducer 54 is disposed in a second-acoustic-field-transducer housing 88. As is seen in FIG. 3, housings 86 and 88 are not rigidly coupled to each other (in fact, they are not coupled to each other at all, in the particular application shown in FIG. 3). Computer processor 29 typically coordinates the displaying of anatomical image 62 and the displaying of acoustic-impedance image 64 on output device 40 (e.g., side by side, or using image fusion) using registration data that register relative dispositions of the housings. (Techniques for registering the relative position and orientation of medical tools are known in the art, for example using mutually-orthogonal RF coils or black-and-white optical patterns.)

For some applications, first acoustic transducer 50 (serving as a first-acoustic-field transducer) is disposed within a first-acoustic-field-transducer housing 84, and first-acoustic-field-transducer housing 84 and second-acoustic-field-transducer housing 88 are rigidly coupled to each other, e.g., by a rigid coupling member 90, which may be, for example, a case within which housings 84 and 88 are disposed (as shown), or a rigid connector disposed between housings 84 and 88.

Reference is made to FIGS. 1-3. Set 80 of acoustic transducers may receive the echo data as echo data containing Doppler-shifted frequencies related to the oscillatory motion of the scatterers in tissue 46. The oscillatory motion results in a time-dependent Doppler shift that oscillates at a frequency that is related to the frequency of first acoustic field 48. Computer processor 29 derives the indication of the acoustic impedance of the tissue by (a) extracting the oscillating time-dependent Doppler shift from the received echo data, (b) converting the extracted Doppler shift into particle-velocity of the first acoustic field, and (c) using the particle-velocity of the first acoustic field to assess the acoustic impedance of the tissue. Techniques described in US 2019/0232090 to Ben-Ezra, which is incorporated herein by reference, may be utilized to facilitate this analysis.

Alternatively or additionally to this procedure for using Doppler-shifted frequencies to determine acoustic impedance and thereby distinguish between different tissues, and still with respect to techniques described with reference to FIGS. 1-3, it is noted that set 80 of acoustic transducers may transmit second acoustic field 56 by transmitting first and second acoustic pulses into the tissue, the first and second pulses being synchronized with first acoustic field 48 and each pulse having a center frequency that is higher than the frequency of first acoustic field 48. Set 80 of acoustic transducers receives respective echoes of each pulse scattering off an oscillating scatterer in tissue 46. Computer processor 29 is derives the indication of acoustic impedance by extracting a time shift between the received echoes that is due to motion of the oscillating scatterer. Based on the extracted time shift, computer processor 29 calculates a displacement amplitude of the oscillating scatterer and assesses the acoustic impedance of the tissue using the calculated displacement amplitude of the first acoustic field. Techniques described in 2021/0045714 to Ben-Ezra, which is incorporated herein by reference, may be utilized to facilitate this analysis.

For some applications, the apparatus and methods described hereinabove facilitate assessment of the spatial distribution of acoustic impedance by using a second acoustic field to measure the velocity (from Doppler shift) and/or the displacement of objects exposed to the first acoustic field. These apparatus and methods may be practiced with the ARFI method described hereinabove, but are different from the ARFI method in several ways. ARFI typically measures elasticity of tissues at very low frequencies (e.g., 1 - 100 Hz), while the techniques described hereinabove with reference to FIGS. 1-3 measure acoustic displacement and acoustic particle-velocity amplitudes that depend on the acoustic impedance of the tissue at higher frequencies (such as many kHz or even above 1 MHz). Thus, the employed frequency ranges being used are typically different by orders of magnitude, but also the physical parameter being measured is different (e.g., acoustic impedance may be measured using the techniques described hereinabove for determining displacement amplitude or Doppler shift). Acoustic impedance Z is the ratio between pressure amplitude p and particle-velocity amplitude U, both of which are intrinsic constituents of the acoustic field, and which in general are independent of attenuation (unlike in the ARFI technique). The particle-velocity amplitude may be derived from the displacement amplitudes for harmonic motion (sinusoidal) by means of the formula:

$$U = w D$$

with U- the velocity amplitude, D - the displacement amplitude and w - the angular velocity. Generally speaking, a change in the acoustic impedance of the medium will be reflected in the velocity amplitude; higher acoustic impedance will result in lower velocity for the same pressure amplitude. Therefore, HIFU imaging response from two adjacent regions (e.g., at the same distance from the transducer), as provided by some applications of the present invention, carries information indicative of the local acoustic impedance value in situ, with higher signal (higher velocity) from the lower impedance region. (As will be appreciated by one skilled in the art having read the present patent application, a given mass of cells or other object will generally have an acoustic impedance Z that depends on frequency Z=Z(w), and knowing this dependence may aid in determining differences between cancerous and noncancerous cells (amongst other uses).) The physical property that is measured is the complex acoustic impedance

$$Z = p / U$$

where p is the pressure and U is the particle velocity as above.

As described hereinabove, acoustic impedance changes may be tracked over time during heating or cooling of tissue. It is noted that the heating time and cooling time for a given incident intensity are affected both by the specific heat of the tissues involved (which may be dependent on whether the tissue is cancerous or not) and also by the perfusion of the tissue involved (i.e., the rate of blood flow through the tissue, with higher perfusion tending to reduce heating), which may generally be higher for cancerous growths in comparison to noncancerous growths of the same tissue type. Other factors affecting the heating response include the heat capacity of the tissue involved, the heat conductivity of the tissue, and the nature of the surrounding tissue and thermal coupling thereto. To determine the heating response of tissue, it is advantageous to track the temperature of the tissue, e.g., using techniques described in an article by Pouch et al., entitled, “In vivo noninvasive temperature measurement by B-mode ultrasound imaging” (J Ultrasound Med. 2010 Nov;29(11):1595-606), or other known ultrasound or non-ultrasound techniques that have been described for the purpose of noninvasive thermometry.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 29. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., computer processor 29) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 29) or other programmable data processing apparatus, create means for implementing the functions/acts specified in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the present patent application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the present application.

Computer processor 29 and the other computer processors described herein are typically hardware devices programmed with computer program instructions to produce a special purpose computer. For example, when programmed to perform the algorithms described herein, the computer processor typically acts as a special purpose computer processor. Typically, the operations described herein that are performed by computer processors transform the physical state of a memory, which is a real physical article, to have a different magnetic polarity, electrical charge, or the like depending on the technology of the memory that is used.

In accordance with some applications of the present invention, there is provided the following list of inventive concepts:
Inventive Concept 1. Apparatus for assessing a characteristic of a tissue, the apparatus comprising:
   a set of one or more acoustic transducers configured to:
      transmit a first acoustic field at a first frequency into the tissue, the first acoustic field generating oscillatory motion at the first frequency of scatterers disposed in the tissue, each scatterer oscillating around a respective equilibrium position,
      transmit a second acoustic field at a second frequency into the tissue, the second frequency higher than the first frequency, and
      receive echo data due to the second acoustic field scattering off an oscillating scatterer in the tissue that is oscillating at the first frequency;
   an output device; and
   at least one computer processor configured to:
      (a) derive an indication of acoustic impedance of the tissue based on the echo data, and
      (b) drive the output device to output an indication of whether the tissue is or may be a tumor, based on the indication of the acoustic impedance of the tissue.
Inventive Concept 2. The apparatus according to Inventive Concept 1, wherein the set of one or more acoustic transducers is configured to perform the steps of transmitting the first acoustic field, transmitting the second acoustic field, and receiving the echo data without therapeutically or diagnostically heating the tissue.
Inventive Concept 3. The apparatus according to Inventive Concept 1, wherein the computer processor is configured to drive the output device to output the indication by driving the output device to display values related to the acoustic impedance of the tissue as an acoustic-impedance image, wherein respective pixel values in the image are indicative of respective acoustic impedance values at different spatial locations within the tissue.
Inventive Concept 4. The apparatus according to Inventive Concept 3, wherein the set of one or more acoustic transducers is further configured to transmit an imaging acoustic field into the tissue, and wherein the computer processor is configured to drive the output device to display an anatomical image of the tissue based on echo data from the imaging acoustic field.
Inventive Concept 5. The apparatus according to Inventive Concept 4, wherein the computer processor is configured to drive the output device to fuse the acoustic-impedance image with the anatomical image.
Inventive Concept 6. The apparatus according to Inventive Concept 4, wherein the set of one or more acoustic transducers is configured to use a single ultrasound transducer for transmitting the first acoustic field, the second acoustic field, and the imaging acoustic field.
Inventive Concept 7. The apparatus according to Inventive Concept 4, wherein:
   the set of one or more acoustic transducers comprises an imaging transducer configured to transmit the imaging acoustic field and a second-acoustic-field transducer configured to transmit the second acoustic field,
   the apparatus further comprises an imaging-transducer housing in which the imaging transducer is disposed, and a second-acoustic-field-transducer housing in which the second-acoustic-field transducer is disposed, the housings not rigidly coupled to each other, and
   the computer processor is configured to coordinate the displaying of the anatomical image and the displaying of the acoustic-impedance image using registration data registering relative dispositions of the housings.
Inventive Concept 8. The apparatus according to Inventive Concept 7, wherein the housings are not coupled to each other.
Inventive Concept 9. The apparatus according to Inventive Concept 7, wherein:
   the set of one or more acoustic transducers comprises a first-acoustic-field transducer configured to transmit the first acoustic field,
   the apparatus further comprises a first-acoustic-field-transducer housing in which the first-acoustic-field transducer is disposed, and
   the first-acoustic-field-transducer housing and the second-acoustic-field-transducer housing are rigidly coupled to each other.
Inventive Concept 10. The apparatus according to Inventive Concept 4, wherein the set of one or more acoustic transducers is configured to use a single ultrasound transducer for transmitting the second acoustic field and the imaging acoustic field.
Inventive Concept 11. The apparatus according to Inventive Concept 10, wherein the set of one or more acoustic transducers comprises a first-acoustic-field transducer configured to transmit the first acoustic field, the first-acoustic-field transducer being distinct from the ultrasound transducer.
Inventive Concept 12. The apparatus according to Inventive Concept 11, wherein the first-acoustic-field transducer is configured to transmit the first acoustic field as ultrasound.
Inventive Concept 13. The apparatus according to any one of Inventive Concepts 1 or 3-12, wherein the set of one or more acoustic transducers is configured to transmit a therapeutic acoustic field into the tissue, subsequently to the driving of the output device and at least in part in response to the derived indication of the acoustic impedance of the tissue, the therapeutic acoustic field having an intensity that is higher than an intensity of the first acoustic field and that is higher than an intensity of the second acoustic field.
Inventive Concept 14. The apparatus according to Inventive Concept 13, wherein the set of one or more acoustic transducers is configured to transmit the therapeutic acoustic field at an intensity that is at least 100 times higher than an intensity of the first acoustic field and that is at least 100 times higher than an intensity of the second acoustic field.
Inventive Concept 15. The apparatus according to Inventive Concept 14, wherein the set of one or more acoustic transducers is configured to transmit the therapeutic acoustic field as high intensity focused ultrasound (HIFU).
Inventive Concept 16. The apparatus according to Inventive Concept 13, wherein the set of one or more acoustic transducers is configured to use a single ultrasound transducer for transmitting the first acoustic field and the therapeutic acoustic field.
Inventive Concept 17. The apparatus according to Inventive Concept 16, wherein the set of one or more acoustic transducers is configured to transmit the second acoustic field using a different ultrasound transducer from that used to transmit the first acoustic field and the therapeutic acoustic field.
Inventive Concept 18. The apparatus according to any one of Inventive Concepts 1 or 3-12, wherein the set of one or more acoustic transducers is configured to transmit the first acoustic field at the first frequency, the first frequency less than 2.5 MHz.
Inventive Concept 19. The apparatus according to Inventive Concept 18, wherein the set of one or more acoustic transducers is configured to transmit the first acoustic field at the first frequency, the first frequency greater than 1 MHz.
Inventive Concept 20. The apparatus according to Inventive Concept 18, wherein the set of one or more acoustic transducers is configured to set the first acoustic field to not be high intensity focused ultrasound (HIFU).
Inventive Concept 21. The apparatus according to Inventive Concept 18, wherein the set of one or more acoustic transducers is configured to transmit the first acoustic field at the first frequency, the first frequency less than 500 kHz.
Inventive Concept 22. The apparatus according to Inventive Concept 21, wherein the set of one or more acoustic transducers is configured to transmit the first acoustic field at the first frequency, the first frequency between 100 kHz and 500 kHz.
Inventive Concept 23. The apparatus according to Inventive Concept 21, wherein the set of one or more acoustic transducers is configured to transmit the first acoustic field at the first frequency, the first frequency between 20 kHz and 100 kHz.
Inventive Concept 24. The apparatus according to Inventive Concept 23, wherein the set of one or more acoustic transducers is configured to transmit the first acoustic field at the first frequency, the first frequency between 50 kHz and 100 kHz.
Inventive Concept 25. The apparatus according to any one of Inventive Concepts 1 or 3-17, wherein the set of one or more acoustic transducers is configured to heat the tissue by at least 1 degree C from a first temperature, by transmitting the first acoustic field.
Inventive Concept 26. The apparatus according to Inventive Concept 25, wherein the set of one or more acoustic transducers is configured to heat the tissue by at least 2° C. from the first temperature, by transmitting the first acoustic field.
Inventive Concept 27. The apparatus according to Inventive Concept 25, wherein the set of one or more acoustic transducers is configured to heat the tissue by less than 5° C. from the first temperature, by transmitting the first acoustic field.
Inventive Concept 28. The apparatus according to Inventive Concept 25, wherein the computer processor is configured to derive the indication of the acoustic impedance at a plurality of time points following initiation of the heating of the tissue, while the tissue is at respective temperatures elevated above the first temperature due to the heating of the tissue.
Inventive Concept 29. The apparatus according to Inventive Concept 28, wherein the computer processor is configured to set a temporal separation between at least one of the plurality of time points and another one of the plurality of time points to be 20-500 milliseconds.
Inventive Concept 30. The apparatus according to Inventive Concept 28, wherein the computer processor is configured to distribute the plurality of time points over at least 5 seconds.
Inventive Concept 31. The apparatus according to Inventive Concept 30, wherein the computer processor is configured to distribute the plurality of time points over 30-120 seconds.
Inventive Concept 32. The apparatus according to Inventive Concept 28, wherein the computer processor is configured to set at least one time point of the plurality of time points to be following termination of the heating of the tissue.
Inventive Concept 33. The apparatus according to Inventive Concept 28, wherein the computer processor is configured to set at least one time point of the plurality of time points to be following initiation of the heating and prior to termination of the heating of the tissue.
Inventive Concept 34. The apparatus according to any one of Inventive Concepts 1 or 3-12 or 18-24, wherein the set of one or more acoustic transducers is configured to inhibit heating of the tissue by controlling an intensity of the first acoustic field.
Inventive Concept 35. The apparatus according to Inventive Concept 34, wherein the set of one or more acoustic transducers is configured to generate the oscillatory motion by transmitting 1-15 cycles of the first acoustic field.
Inventive Concept 36. The apparatus according to Inventive Concept 34, wherein the set of one or more acoustic transducers is configured to control the intensity of the first acoustic field by setting the time between the initiation of successive pulses of ultrasound energy in the first acoustic field to be 20-100 times longer than an average pulse duration of the successive pulses of the ultrasound energy.
Inventive Concept 37. The apparatus according to Inventive Concept 34, wherein the set of one or more acoustic transducers is configured to control the intensity of the first acoustic field by setting the time between the initiation of successive pulses of ultrasound energy in the first acoustic field to be 100-500 times longer than an average pulse duration of the successive pulses of the ultrasound energy.
Inventive Concept 38. The apparatus according to Inventive Concept 34, wherein the computer processor is configured to derive the indication of the acoustic impedance irrespective of any change in the tissue due to any temperature rise of the tissue induced by the first acoustic field.
Inventive Concept 39. The apparatus according to Inventive Concept 34, wherein the set of one or more acoustic transducers is configured to control the intensity by controlling a time-averaged intensity of the first acoustic field.
Inventive Concept 40. The apparatus according to Inventive Concept 39, wherein the set of one or more acoustic transducers is configured to control the time-averaged intensity by setting the time-averaged intensity of the first acoustic field to be less than 720 mW/cm^2.
Inventive Concept 41. The apparatus according to Inventive Concept 34, wherein the set of one or more acoustic transducers is configured to control the intensity by controlling a duty cycle of the first acoustic field.
Inventive Concept 42. The apparatus according to Inventive Concept 41, wherein the set of one or more acoustic transducers is configured to control the duty cycle of the first acoustic field by setting the duty cycle of the first acoustic field to be less than 1%.
Inventive Concept 43. The apparatus according to Inventive Concept 42, wherein the set of one or more acoustic transducers is configured to control the intensity by setting an amplitude of the first acoustic field to be 0.1 - 5 MPa.
Inventive Concept 44. The apparatus according to Inventive Concept 42, wherein the set of one or more acoustic transducers is further configured to control the intensity by setting a pulse repetition frequency (PRF) of the first acoustic field to be 5-50 Hz.
Inventive Concept 45. The apparatus according to Inventive Concept 44, wherein the set of one or more acoustic transducers is configured to set the pulse repetition frequency (PRF) of the first acoustic field to be 10-25 Hz.
Inventive Concept 46. The apparatus according to Inventive Concept 34, wherein the set of one or more acoustic transducers is configured to inhibit heating of the tissue by preventing any therapeutic heating of the tissue.
Inventive Concept 47. The apparatus according to Inventive Concept 46, wherein the set of one or more acoustic transducers is configured to prevent any therapeutic heating of the tissue by preventing any heating of the tissue.
Inventive Concept 48. The apparatus according to Inventive Concept 34, wherein the set of one or more acoustic transducers is configured to prevent any heating of the tissue of more than 2° C.
Inventive Concept 49. The apparatus according to Inventive Concept 48, wherein the set of one or more acoustic transducers is configured to prevent any heating of the tissue of more than 1 degree C.
Inventive Concept 50. The apparatus according to any one of Inventive Concepts 1-49, wherein the set of one or more acoustic transducers is configured to set the second frequency to be 2-50 times higher than the first frequency.
Inventive Concept 51. The apparatus according to Inventive Concept 50, wherein the set of one or more acoustic transducers is configured to set the second frequency to be 2-10 times higher than the first frequency.
Inventive Concept 52. The apparatus according to Inventive Concept 50, wherein the set of one or more acoustic transducers is configured to set the second frequency to be 10-50 times higher than the first frequency.
Inventive Concept 53. The apparatus according to Inventive Concept 50, wherein the set of one or more acoustic transducers is configured to set the second frequency to be 2-12 MHz.
Inventive Concept 54. The apparatus according to any one of Inventive Concepts 1-53, wherein the computer processor is configured to drive the output device to output the indication by driving the output device to display absolute values related to the acoustic impedance of the tissue that are not relative to standard values of acoustic impedance.
Inventive Concept 55. The apparatus according to Inventive Concept 54, wherein the computer processor is configured to drive the output device to display the absolute values by driving the output device to display the absolute values as an acoustic-impedance image, wherein respective pixel values in the acoustic-impedance image are indicative of respective acoustic impedance values at different spatial locations within the tissue.
Inventive Concept 56. The apparatus according to any one of Inventive Concepts 1-53, wherein the computer processor is configured to drive the output device to output the indication by driving the output device to display values related to the acoustic impedance of the tissue that are relative to standard values for acoustic impedance.
Inventive Concept 57. The apparatus according to Inventive Concept 1, wherein the set of one or more acoustic transducers is configured to use a single ultrasound transducer for transmitting the first and second acoustic fields.
Inventive Concept 58. The apparatus according to Inventive Concept 1, wherein the set of one or more acoustic transducers comprises a first ultrasound transducer and a second ultrasound transducer, and wherein the set of one or more acoustic transducers is configured to transmit the first and second acoustic fields using the first and second ultrasound transducers, respectively.
Inventive Concept 59. The apparatus according to Inventive Concept 1, wherein the set of one or more acoustic transducers is configured to transmit the first acoustic field as high intensity focused ultrasound (HIFU).
Inventive Concept 60. The apparatus according to any one of Inventive Concepts 1-59, wherein:
   the set of one or more acoustic transducers is configured to receive the echo data as echo data containing Doppler-shifted frequencies related to the oscillatory motion of the scatterers that results in a time-dependent Doppler shift that oscillates at a frequency that is related to the first frequency, and
   the computer processor is configured to derive the indication of the acoustic impedance of the tissue by (a) extracting the oscillating time-dependent Doppler shift from the received echo data, (b) converting the extracted Doppler shift into particle-velocity of the first acoustic field, and (c) using the particle-velocity of the first acoustic field to assess the acoustic impedance of the tissue.
(A) the set of one or more acoustic transducers is configured to transmit the second acoustic field by:
   transmitting first and second acoustic pulses into the tissue, each pulse having a center frequency that is higher than the first frequency, the first and second pulses being synchronized with the first acoustic field, and
   receiving respective echoes of each pulse scattering off an oscillating scatterer in the tissue, and
(B) the computer processor is configured to derive the indication of acoustic impedance by:
   extracting a time shift between the received echoes that is due to motion of the oscillating scatterer,
   based on the extracted time shift, calculating a displacement amplitude of the oscillating scatterer, and
   using the calculated displacement amplitude of the first acoustic field to assess the acoustic impedance of the tissue.
Inventive Concept 61. The apparatus according to any one of Inventive Concepts 1-59, wherein:
   (A) the set of one or more acoustic transducers is configued to transmit the second acoustic field by:
      transmitting first and second acoustic pulses into the tissue, each pulse having a center fequeny that is higher than the first frequency, the first and second pulses being synchronized with the first acoustic field, and
      recieving respective echoes of each pulse scattering off an oscillating scatterer in the tisuue, and
   (B) the computer processor is configured to erive the indication of acoustic impedance by:
      extracting a time shift between the receivved echoes that is due to motion of the oscillating scatterer,
      based on the extracted time shift, calculating a displacement amplitude of the oscillating scatterer, and
      using the calculated displacement amplitude of the first acoustic field to assess the acoustic impedance of the tissue.
Inventive Concept 62. A method for assessing a characteristic of a tissue, the methodd comprising:
   transmitting a first acoustic field at a first frequency into the tissue, the first acoustic field generating oscillatory motion at the first frequency of scatterers disposed in the tissue, each scatterer oscillating around a respective equilibrium position;
   transmitting a second acoustic field at a second frequency into the tissue, the second frequency higher than the first frequency;
   receiving echo data due to the second acoustic field scattering off an oscillating scatterer in the tissue that is oscillating at the first frequency; and
   using at least one computer processor:
      (a) deriving an indication of acoustic impedance of the tissue based on the echo data, and
      (b) driving an output device to output an indication of whether the tissue is or may be a tumor, based on the indication of the acoustic impedance of the tissue.
Inventive Concept 63. The method according to Inventive Concept 62, wherein performing the steps of transmitting the first acoustic field, transmitting the second acoustic field, and receiving the echo data do not comprise therapeutically or diagnostically heating the tissue.
Inventive Concept 64. The method according to Inventive Concept 62, further comprising performing a biopsy subsequently to driving the output device to output the indication of whether the tissue is or may be a tumor.
Inventive Concept 65. The method according to Inventive Concept 64, wherein performing the biopsy comprises performing the biopsy under ultrasound guidance in a same procedure in which the first and second acoustic fields are transmitted.
Inventive Concept 66. The method according to Inventive Concept 62, wherein driving the output device to output the indication comprises driving the output device to display absolute values related to the acoustic impedance of the tissue that are not relative to standard values of acoustic impedance.
Inventive Concept 67. The method according to Inventive Concept 66, wherein driving the output device to display the absolute values comprises driving the output device to display the absolute values as an acoustic-impedance image, wherein respective pixel values in the acoustic-impedance image are indicative of respective acoustic impedance values at different spatial locations within the tissue.
Inventive Concept 68. The method according to Inventive Concept 62, wherein driving the output device to output the indication comprises driving the output device to display values related to the acoustic impedance of the tissue that are relative to standard values for acoustic impedance.
Inventive Concept 69. The method according to Inventive Concept 62, wherein driving the output device to output the indication comprises driving the output device to display values related to the acoustic impedance of the tissue as an acoustic-impedance image, wherein respective pixel values in the image are indicative of respective acoustic impedance values at different spatial locations within the tissue.
Inventive Concept 70. The method according to Inventive Concept 69, further comprising transmitting an imaging acoustic field into the tissue, and wherein driving the output device further comprises driving the output device to display an anatomical image of the tissue based on echo data from the imaging acoustic field.
Inventive Concept 71. The method according to Inventive Concept 70, wherein driving the output device comprises fusing the acoustic-impedance image with the anatomical image.
Inventive Concept 72. The method according to Inventive Concept 70, wherein transmitting the first acoustic field, transmitting the second acoustic field, and transmitting the imaging acoustic field comprises transmitting the first acoustic field, the second acoustic field, and the imaging acoustic field using a same ultrasound transducer.
Inventive Concept 73. The method according to Inventive Concept 70:
   wherein transmitting the imaging acoustic field and transmitting the second acoustic field comprises transmitting the imaging acoustic field and transmitting the second acoustic field using separate ultrasound transducers disposed respectively in an imaging-transducer housing and in a second-acoustic-field-transducer housing, the housings not rigidly coupled to each other, and
   further comprising coordinating the displaying of the anatomical image and the displaying of the acoustic-impedance image using registration data registering relative dispositions of the housings.
Inventive Concept 74. The method according to Inventive Concept 73, wherein the housings are not coupled to each other.
Inventive Concept 75. The method according to Inventive Concept 73, wherein transmitting the first acoustic field comprises transmitting the first acoustic field using a first-acoustic-field transducer disposed in a first-acoustic-field-transducer housing, and wherein the first-acoustic-field-transducer housing and the second-acoustic-field-transducer housing are rigidly coupled to each other.
Inventive Concept 76. The method according to Inventive Concept 70, wherein transmitting the second acoustic field and transmitting the imaging acoustic field comprises transmitting the second acoustic field and transmitting the imaging acoustic field using a same ultrasound transducer.
Inventive Concept 77. The method according to Inventive Concept 76, wherein transmitting the first acoustic field comprises transmitting the first acoustic field using an acoustic transducer that is not the same ultrasound transducer used to transmit the second acoustic field and the imaging acoustic field.
Inventive Concept 78. The method according to Inventive Concept 77, wherein transmitting the first acoustic field using the acoustic transducer comprises transmitting the first acoustic field using another ultrasound transducer.
Inventive Concept 79. The method according to Inventive Concept 62, further comprising, subsequently to the driving of the output device and at least in part in response to the derived indication of the acoustic impedance of the tissue, transmitting a therapeutic acoustic field into the tissue, the therapeutic acoustic field having an intensity that is higher than an intensity of the first acoustic field and that is higher than an intensity of the second acoustic field.
Inventive Concept 80. The method according to Inventive Concept 79, wherein transmitting the therapeutic acoustic field comprises transmitting the therapeutic acoustic field at an intensity that is at least 100 times higher than an intensity of the first acoustic field and that is at least 100 times higher than an intensity of the second acoustic field.
Inventive Concept 81. The method according to Inventive Concept 80, wherein transmitting the therapeutic acoustic field comprises configuring the therapeutic acoustic field to be high intensity focused ultrasound (HIFU).
Inventive Concept 82. The method according to Inventive Concept 79, wherein transmitting the first acoustic field and transmitting the therapeutic acoustic field comprises transmitting the first acoustic field and transmitting the therapeutic acoustic field using a same ultrasound transducer.
Inventive Concept 83. The method according to Inventive Concept 82, wherein transmitting the second acoustic field comprises transmitting the second acoustic field using a different ultrasound transducer from that used to transmit the first acoustic field and the therapeutic acoustic field.
Inventive Concept 84. The method according to Inventive Concept 62, wherein transmitting the first acoustic field comprises transmitting the first acoustic field at the first frequency, the first frequency less than 2.5 MHz.
Inventive Concept 85. The method according to Inventive Concept 84, wherein transmitting the first acoustic field comprises transmitting the first acoustic field at the first frequency, the first frequency greater than 1 MHz.
Inventive Concept 86. The method according to Inventive Concept 84, wherein transmitting the first acoustic field comprises configuring the first acoustic field to not be high intensity focused ultrasound (HIFU).
Inventive Concept 87. The method according to Inventive Concept 84, wherein transmitting the first acoustic field comprises transmitting the first acoustic field at the first frequency, the first frequency less than 500 kHz.
Inventive Concept 88. The method according to Inventive Concept 87, wherein transmitting the first acoustic field comprises transmitting the first acoustic field at the first frequency, the first frequency between 100 kHz and 500 kHz.
Inventive Concept 89. The method according to Inventive Concept 87, wherein transmitting the first acoustic field comprises transmitting the first acoustic field at the first frequency, the first frequency between 20 kHz and 100 kHz.
Inventive Concept 90. The method according to Inventive Concept 89, wherein transmitting the first acoustic field comprises transmitting the first acoustic field at the first frequency, the first frequency between 50 kHz and 100 kHz.
Inventive Concept 91. The method according to Inventive Concept 62, wherein transmitting the first acoustic field comprises heating the tissue by at least 1 degree C from a first temperature.
Inventive Concept 92. The method according to Inventive Concept 91, wherein heating the tissue comprises heating the tissue by at least 2° C. from the first temperature.
Inventive Concept 93. The method according to Inventive Concept 91, wherein heating the tissue comprises heating the tissue by less than 5° C. from the first temperature.
Inventive Concept 94. The method according to Inventive Concept 91, wherein deriving the indication of the acoustic impedance comprises deriving the indication of the acoustic impedance at a plurality of time points following initiation of the heating of the tissue, while the tissue is at respective temperatures elevated above the first temperature due to the heating of the tissue.
Inventive Concept 95. The method according to Inventive Concept 94, wherein at least one of the plurality of time points is separated from another one of the plurality of time points by 20-500 milliseconds.
Inventive Concept 96. The method according to Inventive Concept 94, wherein deriving the indication of the acoustic impedance at the plurality of time points comprises deriving the indication of the acoustic impedance at the plurality of time points, the plurality of time points distributed over at least 5 seconds.
Inventive Concept 97. The method according to Inventive Concept 96, wherein deriving the indication of the acoustic impedance at the plurality of time points comprises deriving the indication of the acoustic impedance at the plurality of time points, the plurality of time points distributed over 30-120 seconds.
Inventive Concept 98. The method according to Inventive Concept 94, wherein deriving the indication of the acoustic impedance at the plurality of time points following initiation of the heating of the tissue comprises deriving the indication of the acoustic impedance at at least one time point that is following termination of the heating of the tissue.
Inventive Concept 99. The method according to Inventive Concept 94, wherein deriving the indication of the acoustic impedance at the plurality of time points following initiation of the heating of the tissue comprises deriving the indication of the acoustic impedance at at least one time point that is following initiation of the heating and prior to termination of the heating of the tissue.
Inventive Concept 100. The method according to Inventive Concept 62, wherein transmitting the first acoustic field comprises inhibiting heating of the tissue by controlling an intensity of the first acoustic field.
Inventive Concept 101. The method according to Inventive Concept 100, wherein transmitting the first acoustic field comprises generating the oscillatory motion by transmitting 1-15 cycles of the first acoustic field.
Inventive Concept 102. The method according to Inventive Concept 100, wherein controlling the intensity of the first acoustic field comprises setting the time between the initiation of successive pulses of ultrasound energy in the first acoustic field to be 20-100 times longer than an average pulse duration of the successive pulses of the ultrasound energy.
Inventive Concept 103. The method according to Inventive Concept 100, wherein controlling the intensity of the first acoustic field comprises setting the time between the initiation of successive pulses of ultrasound energy in the first acoustic field to be 100-500 times longer than an average pulse duration of the successive pulses of the ultrasound energy.
Inventive Concept 104. The method according to Inventive Concept 100, wherein the step of deriving the indication of the acoustic impedance is performed irrespective of any change in the tissue due to any temperature rise of the tissue induced by the first acoustic field.
Inventive Concept 105. The method according to Inventive Concept 100, wherein controlling the intensity comprises controlling a time-averaged intensity of the first acoustic field.
Inventive Concept 106. The method according to Inventive Concept 105, wherein controlling the time-averaged intensity comprises setting the time-averaged intensity of the first acoustic field to be less than 720 mW/cm^2.
Inventive Concept 107. The method according to Inventive Concept 100, wherein controlling the intensity comprises controlling a duty cycle of the first acoustic field.
Inventive Concept 108. The method according to Inventive Concept 107, wherein controlling the duty cycle of the first acoustic field comprises setting the duty cycle of the first acoustic field to be less than 1%.
Inventive Concept 109. The method according to Inventive Concept 108, wherein controlling the intensity comprises setting an amplitude of the first acoustic field to be 0.1 -5 MPa.
Inventive Concept 110. The method according to Inventive Concept 108, wherein controlling the intensity further comprises setting a pulse repetition frequency (PRF) of the first acoustic field to be 5-50 Hz.
Inventive Concept 111. The method according to Inventive Concept 110, wherein setting the PRF comprises setting the pulse repetition frequency (PRF) of the first acoustic field to be 10-25 Hz.
Inventive Concept 113. The method according to Inventive Concept 112, wherein preventing any therapeutic heating of the tissue comprises preventing any heating of the tissue.
Inventive Concept 114. The method according to Inventive Concept 100, wherein inhibiting heating of the tissue by controlling the intensity of the first acoustic field comprises preventing any heating of the tissue of more than 2° C.
Inventive Concept 115. The method according to Inventive Concept 114, wherein preventing any heating of the tissue of more than 2° C. comprises preventing any heating of the tissue of more than 1 degree C.
Inventive Concept 116. The method according to Inventive Concept 62, wherein transmitting the second acoustic field at the second frequency comprises configuring the second frequency to be 2-50 times higher than the first frequency.
Inventive Concept 117. The method according to Inventive Concept 116, wherein the second frequency is 2-10 times higher than the first frequency.
Inventive Concept 118. The method according to Inventive Concept 116, wherein the second frequency is 10-50 times higher than the first frequency.
Inventive Concept 119. The method according to Inventive Concept 116, wherein the second frequency is 2-12 MHz.
Inventive Concept 120. The method according to Inventive Concept 62, wherein transmitting the first and second acoustic fields comprises transmitting the first and second acoustic fields using a same ultrasound transducer.
Inventive Concept 121. The method according to Inventive Concept 62, wherein transmitting the first and second acoustic fields comprises transmitting the first and second acoustic fields using respective first and second ultrasound transducers.
Inventive Concept 122. The method according to Inventive Concept 62, wherein transmitting the first acoustic field comprises configuring the first acoustic field to be high intensity focused ultrasound (HIFU).
Inventive Concept 123. The method according to Inventive Concept 62, wherein:
   receiving the echo data comprises receiving echo data containing Doppler-shifted frequencies related to the oscillatory motion of the scatterers that results in a time-dependent Doppler shift that oscillates at a frequency that is related to the first frequency, and
   deriving the indication of the acoustic impedance of the tissue comprises (a) extracting the oscillating time-dependent Doppler shift from the received echo data, (b) converting the extracted Doppler shift into particle-velocity of the first acoustic field, and (c) using the particle-velocity of the first acoustic field to assess the acoustic impedance of the tissue.
Inventive Concept 124. The method according to Inventive Concept 62, wherein:
   (A) transmitting the second acoustic field comprises:
      transmitting first and second acoustic pulses into the tissue, each pulse having a center frequency that is higher than the first frequency, the first and second pulses being synchronized with the first acoustic field; and
      receiving respective echoes of each pulse scattering off an oscillating scatterer in the tissue, and
   (B) deriving the indication of acoustic impedance comprises:
      extracting a time shift between the received echoes that is due to motion of the oscillating scatterer;
      based on the extracted time shift, calculating a displacement amplitude of the oscillating scatterer; and
      using the calculated displacement amplitude of the first acoustic field to assess the acoustic impedance of the tissue.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for assessing a characteristic of a tissue, the apparatus comprising:
a set of one or more acoustic transducers configured to:
transmit a first acoustic field at a first frequency into the tissue, the first acoustic field generating oscillatory motion at the first frequency of scatterers disposed in the tissue, each scatterer oscillating around a respective equilibrium position,
transmit a second acoustic field at a second frequency into the tissue, the second frequency higher than the first frequency, and
receive echo data due to the second acoustic field scattering off an oscillating scatterer in the tissue that is oscillating at the first frequency;
an output device; and
at least one computer processor configured to:
(a) derive an indication of acoustic impedance of the tissue based on the echo data, and
(b) drive the output device to output an indication of whether the tissue is or may be a tumor, based on the indication of the acoustic impedance of the tissue.

2. The apparatus according to claim 1, wherein the set of one or more acoustic transducers is configured to perform the steps of transmitting the first acoustic field, transmitting the second acoustic field, and receiving the echo data without therapeutically or diagnostically heating the tissue.

3. The apparatus according to claim 1, wherein the computer processor is configured to drive the output device to output the indication by driving the output device to display values related to the acoustic impedance of the tissue as an acoustic-impedance image, wherein respective pixel values in the image are indicative of respective acoustic impedance values at different spatial locations within the tissue.

4. The apparatus according to claim 3, wherein the set of one or more acoustic transducers is further configured to transmit an imaging acoustic field into the tissue, and wherein the computer processor is configured to drive the output device to display an anatomical image of the tissue based on echo data from the imaging acoustic field.

5. The apparatus according to claim 4, wherein the computer processor is configured to drive the output device to fuse the acoustic-impedance image with the anatomical image.

6. The apparatus according to claim 4, wherein the set of one or more acoustic transducers is configured to use a single ultrasound transducer for transmitting the first acoustic field, the second acoustic field, and the imaging acoustic field.

7. The apparatus according to claim 4, wherein:
the set of one or more acoustic transducers comprises an imaging transducer configured to transmit the imaging acoustic field and a second-acoustic-field transducer configured to transmit the second acoustic field,
the apparatus further comprises an imaging-transducer housing in which the imaging transducer is disposed, and a second-acoustic-field-transducer housing in which the second-acoustic-field transducer is disposed, the housings not rigidly coupled to each other, and
the computer processor is configured to coordinate the displaying of the anatomical image and the displaying of the acoustic-impedance image using registration data registering relative dispositions of the housings.

8. The apparatus according to claim 7, wherein the housings are not coupled to each other.

9. The apparatus according to claim 7, wherein:
the set of one or more acoustic transducers comprises a first-acoustic-field transducer configured to transmit the first acoustic field,
the apparatus further comprises a first-acoustic-field-transducer housing in which the first-acoustic-field transducer is disposed, and
the first-acoustic-field-transducer housing and the second-acoustic-field-transducer housing are rigidly coupled to each other.

10. The apparatus according to claim 4, wherein the set of one or more acoustic transducers is configured to use a single ultrasound transducer for transmitting the second acoustic field and the imaging acoustic field.

11. The apparatus according to claim 10, wherein the set of one or more acoustic transducers comprises a first-acoustic-field transducer configured to transmit the first acoustic field, the first-acoustic-field transducer being distinct from the ultrasound transducer.

12. The apparatus according to claim 11, wherein the first-acoustic-field transducer is configured to transmit the first acoustic field as ultrasound.

13. The apparatus according to any one of claims 1 or 3-12, wherein the set of one or more acoustic transducers is configured to transmit a therapeutic acoustic field into the tissue, subsequently to the driving of the output device and at least in part in response to the derived indication of the acoustic impedance of the tissue, the therapeutic acoustic field having an intensity that is higher than an intensity of the first acoustic field and that is higher than an intensity of the second acoustic field.

14. The apparatus according to claim 13, wherein the set of one or more acoustic transducers is configured to transmit the therapeutic acoustic field at an intensity that is at least 100 times higher than an intensity of the first acoustic field and that is at least 100 times higher than an intensity of the second acoustic field.

15. The apparatus according to claim 14, wherein the set of one or more acoustic transducers is configured to transmit the therapeutic acoustic field as high intensity focused ultrasound (HIFU).

16. The apparatus according to claim 13, wherein the set of one or more acoustic transducers is configured to use a single ultrasound transducer for transmitting the first acoustic field and the therapeutic acoustic field.

17. The apparatus according to claim 16, wherein the set of one or more acoustic transducers is configured to transmit the second acoustic field using a different ultrasound transducer from that used to transmit the first acoustic field and the therapeutic acoustic field.

18. The apparatus according to any one of claims 1 or 3-12, wherein the set of one or more acoustic transducers is configured to transmit the first acoustic field at the first frequency, the first frequency less than 2.5 MHz.

19. The apparatus according to claim 18, wherein the set of one or more acoustic transducers is configured to transmit the first acoustic field at the first frequency, the first frequency greater than 1 MHz.

20. The apparatus according to claim 18, wherein the set of one or more acoustic transducers is configured to set the first acoustic field to not be high intensity focused ultrasound (HIFU).

21. The apparatus according to claim 18, wherein the set of one or more acoustic transducers is configured to transmit the first acoustic field at the first frequency, the first frequency less than 500 kHz.

22. The apparatus according to claim 21, wherein the set of one or more acoustic transducers is configured to transmit the first acoustic field at the first frequency, the first frequency between 100 kHz and 500 kHz.

23. The apparatus according to claim 21, wherein the set of one or more acoustic transducers is configured to transmit the first acoustic field at the first frequency, the first frequency between 20 kHz and 100 kHz.

24. The apparatus according to claim 23, wherein the set of one or more acoustic transducers is configured to transmit the first acoustic field at the first frequency, the first frequency between 50 kHz and 100 kHz.

25. The apparatus according to any one of claims 1 or 3-17, wherein the set of one or more acoustic transducers is configured to heat the tissue by at least 1 degree C from a first temperature, by transmitting the first acoustic field.

26. The apparatus according to claim 25, wherein the set of one or more acoustic transducers is configured to heat the tissue by at least 2° C. from the first temperature, by transmitting the first acoustic field.

27. The apparatus according to claim 25, wherein the set of one or more acoustic transducers is configured to heat the tissue by less than 5° C. from the first temperature, by transmitting the first acoustic field.

28. The apparatus according to claim 25, wherein the computer processor is configured to derive the indication of the acoustic impedance at a plurality of time points following initiation of the heating of the tissue, while the tissue is at respective temperatures elevated above the first temperature due to the heating of the tissue.

29. The apparatus according to claim 28, wherein the computer processor is configured to set a temporal separation between at least one of the plurality of time points and another one of the plurality of time points to be 20-500 milliseconds.

30. The apparatus according to claim 28, wherein the computer processor is configured to distribute the plurality of time points over at least 5 seconds.

31. The apparatus according to claim 30, wherein the computer processor is configured to distribute the plurality of time points over 30-120 seconds.

32. The apparatus according to claim 28, wherein the computer processor is configured to set at least one time point of the plurality of time points to be following termination of the heating of the tissue.

33. The apparatus according to claim 28, wherein the computer processor is configured to set at least one time point of the plurality of time points to be following initiation of the heating and prior to termination of the heating of the tissue.

34. The apparatus according to any one of claims 1 or 3-12 or 18-24, wherein the set of one or more acoustic transducers is configured to inhibit heating of the tissue by controlling an intensity of the first acoustic field.

35. The apparatus according to claim 34, wherein the set of one or more acoustic transducers is configured to generate the oscillatory motion by transmitting 1-15 cycles of the first acoustic field.

36. The apparatus according to claim 34, wherein the set of one or more acoustic transducers is configured to control the intensity of the first acoustic field by setting the time between the initiation of successive pulses of ultrasound energy in the first acoustic field to be 20-100 times longer than an average pulse duration of the successive pulses of the ultrasound energy.

37. The apparatus according to claim 34, wherein the set of one or more acoustic transducers is configured to control the intensity of the first acoustic field by setting the time between the initiation of successive pulses of ultrasound energy in the first acoustic field to be 100-500 times longer than an average pulse duration of the successive pulses of the ultrasound energy.

38. The apparatus according to claim 34, wherein the computer processor is configured to derive the indication of the acoustic impedance irrespective of any change in the tissue due to any temperature rise of the tissue induced by the first acoustic field.

39. The apparatus according to claim 34, wherein the set of one or more acoustic transducers is configured to control the intensity by controlling a time-averaged intensity of the first acoustic field.

40. The apparatus according to claim 39, wherein the set of one or more acoustic transducers is configured to control the time-averaged intensity by setting the time-averaged intensity of the first acoustic field to be less than 720 mW/cm^2.

41. The apparatus according to claim 34, wherein the set of one or more acoustic transducers is configured to control the intensity by controlling a duty cycle of the first acoustic field.

42. The apparatus according to claim 41, wherein the set of one or more acoustic transducers is configured to control the duty cycle of the first acoustic field by setting the duty cycle of the first acoustic field to be less than 1%.

43. The apparatus according to claim 42, wherein the set of one or more acoustic transducers is configured to control the intensity by setting an amplitude of the first acoustic field to be 0.1 - 5 MPa.

44. The apparatus according to claim 42, wherein the set of one or more acoustic transducers is further configured to control the intensity by setting a pulse repetition frequency (PRF) of the first acoustic field to be 5-50 Hz.

45. The apparatus according to claim 44, wherein the set of one or more acoustic transducers is configured to set the pulse repetition frequency (PRF) of the first acoustic field to be 10-25 Hz.

46. The apparatus according to claim 34, wherein the set of one or more acoustic transducers is configured to inhibit heating of the tissue by preventing any therapeutic heating of the tissue.

47. The apparatus according to claim 46, wherein the set of one or more acoustic transducers is configured to prevent any therapeutic heating of the tissue by preventing any heating of the tissue.

48. The apparatus according to claim 34, wherein the set of one or more acoustic transducers is configured to prevent any heating of the tissue of more than 2° C.

49. The apparatus according to claim 48, wherein the set of one or more acoustic transducers is configured to prevent any heating of the tissue of more than 1 degree C.

50. The apparatus according to any one of claims 1-49, wherein the set of one or more acoustic transducers is configured to set the second frequency to be 2-50 times higher than the first frequency.

51. The apparatus according to claim 50, wherein the set of one or more acoustic transducers is configured to set the second frequency to be 2-10 times higher than the first frequency.

52. The apparatus according to claim 50, wherein the set of one or more acoustic transducers is configured to set the second frequency to be 10-50 times higher than the first frequency.

53. The apparatus according to claim 50, wherein the set of one or more acoustic transducers is configured to set the second frequency to be 2-12 MHz.

54. The apparatus according to any one of claims 1-53, wherein the computer processor is configured to drive the output device to output the indication by driving the output device to display absolute values related to the acoustic impedance of the tissue that are not relative to standard values of acoustic impedance.

55. The apparatus according to claim 54, wherein the computer processor is configured to drive the output device to display the absolute values by driving the output device to display the absolute values as an acoustic-impedance image, wherein respective pixel values in the acoustic-impedance image are indicative of respective acoustic impedance values at different spatial locations within the tissue.

56. The apparatus according to any one of claims 1-53, wherein the computer processor is configured to drive the output device to output the indication by driving the output device to display values related to the acoustic impedance of the tissue that are relative to standard values for acoustic impedance.

57. The apparatus according to claim 1, wherein the set of one or more acoustic transducers is configured to use a single ultrasound transducer for transmitting the first and second acoustic fields.

58. The apparatus according to claim 1, wherein the set of one or more acoustic transducers comprises a first ultrasound transducer and a second ultrasound transducer, and wherein the set of one or more acoustic transducers is configured to transmit the first and second acoustic fields using the first and second ultrasound transducers, respectively.

59. The apparatus according to claim 1, wherein the set of one or more acoustic transducers is configured to transmit the first acoustic field as high intensity focused ultrasound (HIFU).

60. The apparatus according to any one of claims 1-59, wherein:
the set of one or more acoustic transducers is configured to receive the echo data as echo data containing Doppler-shifted frequencies related to the oscillatory motion of the scatterers that results in a time-dependent Doppler shift that oscillates at a frequency that is related to the first frequency, and
the computer processor is configured to derive the indication of the acoustic impedance of the tissue by (a) extracting the oscillating time-dependent Doppler shift from the received echo data, (b) converting the extracted Doppler shift into particle-velocity of the first acoustic field, and (c) using the particle-velocity of the first acoustic field to assess the acoustic impedance of the tissue.

61. The apparatus according to any one of claims 1-59, wherein:
(A) the set of one or more acoustic transducers is configured to transmit the second acoustic field by:
transmitting first and second acoustic pulses into the tissue, each pulse having a center frequency that is higher than the first frequency, the first and second pulses being synchronized with the first acoustic field, and
receiving respective echoes of each pulse scattering off an oscillating scatterer in the tissue, and
(B) the computer processor is configured to derive the indication of acoustic impedance by:
extracting a time shift between the received echoes that is due to motion of the oscillating scatterer,
based on the extracted time shift, calculating a displacement amplitude of the oscillating scatterer, and
using the calculated displacement amplitude of the first acoustic field to assess the acoustic impedance of the tissue.
